(19) United States
(12) Patent Application Publication
(10) Pub. No.: US 2012/0141577 A1
(43) Pub. Date: Jun. 7, 2012

(54) LINEAR EXPRESSION CASSETTE VACCINES

(75) Inventors: Marston Manthorpe, San Diego, CA (US); Adrian Vilalta, San Diego, CA (US); Alain Rolland, San Diego, CA (US)

(73) Assignee: Vical Incorporated, San Diego, CA (US)

(21) Appl. No.: 11/949,745

(22) Filed: Dec. 3, 2007

Related U.S. Application Data
(60) Provisional application No. 60/868,496, filed on Dec. 4, 2006.

(51) Int. Cl. A61K 9/127; A61P 37/04; A61K 47/00; A61K 39/00; C07H 21/04; A61K 31/7088

(52) U.S. Cl. 424/450; 536/24.1; 536/23.4; 514/44 R; 424/278.1; 424/184.1

(57) ABSTRACT

The disclosure relates to a linear expression cassette (LEC) as a nucleic acid based vector for producing a gene product of interest. Methods of preparing a disclosed LEC, as well as methods for its use to express a gene product in a subject, are also described. An LEC may be used in an animal or human subject to produce a therapeutic and/or immune response in the subject, such as an immunized state.

### CROSS REFERENCE TO OTHER APPLICATIONS

This application claims benefit of priority to provisional U.S. Application No. 60/868,496, filed Dec. 4, 2006, the disclosure of which is fully incorporated herein by reference.

### STATEMENT AS TO RIGHTS TO INVENTIONS MADE UNDER FEDERALLY SPONSORED RESEARCH AND DEVELOPMENT

Some work described herein was partially funded by DARPA grant W911NF-05-1-0545. The U.S. Federal Government has certain rights in the disclosed invention.

### FIELD OF THE DISCLOSURE

This disclosure relates to a linear expression cassette (LEC) as a nucleic acid based expression vector as well as methods of preparing LECs. This disclosure also includes use of LECs to express an encoded gene product in a subject, such as in an animal or human subject to produce a therapeutic and/or an immune response therein. The range of LECs thus includes their use to produce a therapeutic and/or an immunotherapeutic response or immunized state in the subject.

### BACKGROUND OF THE DISCLOSURE

DNA vaccines have proven effective in a growing number of infectious disease indications in several animal models including mice (Chen et al. “Protection and antibody responses in different strains of mouse immunized with plasmid DNAs encoding influenza virus haemagglutinin, neuraminidase and nucleoprotein.” J Gen Virol 1999, 80 (Pt 10), 2559-2564; Davis et al. “West Nile virus recombinant DNA vaccine protects mouse and horse from virus challenge and expresses in vitro a noninfectious recombinant antigen that can be used in enzyme-linked immunosorbent assays.” J Virol 2001, 75(9), 4040-4047; Margalith & Vilalta, “Sustained protective rabies neutralizing antibody titers after administration of cationic lipid-formulated pDNA vaccine.” Genet Vaccines Ther 2006, 4, 2), rabbits (Hermanson et al. “A cationic lipid-formulated plasmid DNA vaccine confers sustained antibody-mediated protection against aerosolized anthrax spores.” Proc Natl Acad Sci USA 2004, 101(37), 13601-13606), dogs (Bahloul et al. “Field trials of a very potent rabies DNA vaccine which induced long lasting virus neutralizing antibodies and protection in dogs in experimental conditions.” Vaccine 2006, 24(8), 1063-1072; Lodmell et al. “Canine rabies DNA vaccination: a single-dose intradermal injection into ear pinnae elicits elevated and persistent levels of neutralizing antibody.” Vaccine 2003, 21(25-26), 3998-4002; Lodmell et al. “One-time intradermal DNA vaccination in ear pinnae one year prior to infection protects dogs against rabies virus.” Vaccine 2006, 24(4), 412-416), monkeys (Lodmell et al. “DNA immunization protects nonhuman primates against rabies virus.” Nat Med 1998, 4(8), 949-952), ferrets (Webster et al. “Protection of ferrets against influenza challenge with a DNA vaccine to the haemagglutinin.” Vaccine 1994, 12(16), 1495-1498), fish (Corbeil et al. “Fish DNA vaccine against infectious hematopoietic necrosis virus: efficacy of various routes of immunisation.” Fish Shellfish Immunol 2000, 10(8), 711-723; Kurath, G. “Overview of recent DNA vaccine development for fish.” Dev Biol (Basel) 2005, 121, 201-213) and horses (Davis et al.; Fischer et al. “Rabies DNA vaccine in the horse: strategies to improve serological responses.” Vaccine 2003, 21(31), 4593-4596). Currently, DNA vaccines are being tested for safety and efficacy in humans for a wide range of indications including HIV, malaria, West Nile Virus, CMV, SARS, and Ebola amongst others.

As a further example, protective responses to influenza A after plasmid DNA (pDNA) vaccination in mice were first reported in 1993 (Ulmer et al. “Heterologous protection against influenza by injection of DNA encoding a viral protein.” Science 1993, 259(5102), 1745-1749; Robinson et al. “Protection against a lethal influenza virus challenge by immunization with a haemagglutinin-expressing plasmid DNA.” Vaccine 1993, 11(9), 957-960; Montgomery et al. “Heterologous and homologous protection against influenza A by DNA vaccination: optimization of DNA vectors.” DNA Cell Biol 1993, 12(9), 777-783).

A DNA vaccine against influenza is desirable. Influenza, a highly contagious illness of the respiratory tract, is caused by RNA viruses of the Orthomyxoviridae family (Knipe & Howley). There are three main types of influenza viruses, namely A, B and C. Structurally, influenza A and B are similar while influenza C presents a different pattern of surface antigens. Major outbreaks of influenza are associated with influenza A or B while infection with type C virus is associated with minor symptoms.

According to the Centers for Disease Control and Prevention, five to 20% of the U.S. population gets influenza every year, with about 36,000 deaths annually due to complications from the infection. Influenza is among the top seven leading causes of death in the U.S. despite over 60 years of licensed influenza vaccine availability; still, the most effective protection against influenza infection is vaccination. Small periodic changes in the virus surface antigens (antigenic drift) require the development of new vaccines every season. Moreover, major changes in the virus surface proteins (antigenic shift) can result in highly virulent strains that have the potential to cause a pandemic. Outbreaks of influenza in animals increase the chances of a pandemic, through the reshuffling of animal and human influenza virus genomes resulting in a new virus strain. Currently, the spread of a deadly strain of avian influenza virus (H5N1) has raised concerns of a potential pandemic and has therefore triggered a re-evaluation of vaccination strategies. It has become clear that successful containment of an outbreak of a highly virulent influenza strain will require fast manufacture of large quantities of vaccines.

Published data indicate that vaccination with pDNA encoding either Nucleocapsid (NP) (Ulmer et al.), Neuroaminidase (NA) or Hemagglutinin (HA) (Chen et al.) of influenza can induce protective responses in mice, although protection might be somewhat dependent on the strain of mice. Reports also indicate that both ferrets and non-human primates can be successfully vaccinated using influenza genes expressed from pDNA (Webster et al.; Donnelly et al. “Preclinical efficacy of a prototype DNA vaccine: enhanced protection against antigenic drift in influenza virus.” Nat Med 1995, 1(6), 583-587; Donnelly et al. “Further protection against antigenic drift of influenza virus in a ferret model by DNA vaccination.” Vaccine 1997, 15(8), 865-868). Although the consensus in the field is that antibody responses to HA are essential (Knipe, D. M. & Howley, P. M. Fields Virology, Lippincott Williams and Wilkins, Philadelphia, 2001), inclusion of other antigens such as the highly conserved NP, M1 and M2 proteins could provide protection against genetic drift through the development of cellular immunity.

Moreover, protection against influenza A infection requires strong humoral responses. Intramuscular injection of pDNA in PBS has been shown to elicit potent cellular but relatively low antibody responses. Therefore, the use of formulations that drive robust B-cell responses would be expected to improve the performance of nucleic acid-based influenza vaccines. Complexing immunogen-encoding DNA with cationic lipid systems, such as DMRIE:DOPE and Vaxfectin™ offers a potential enhancement to humoral response elicited by DNA in PBS. For instance, Hartikka and co-workers (“Vaxfectin enhances the humoral immune response to plasmid DNA-encoded antigens.” Vaccine 2001, 19(15-16), 1911-1923) obtained a 20-fold increase in antibody titers against influenza A NP when formulating pDNA with Vaxfectin™ compared to titers obtained with pDNA in PBS. The enhancement of immune responses by Vaxfectin™ formulations is more pronounced when administering low doses of DNA (Margalith & Vilalta) which could be translated into significant dose-sparing. Cationic lipid formulations can provide a significant enhancement of protective immune responses as demonstrated by Hermanson and co-workers (Hermanson et al.) who reported the successful protection of rabbits against a lethal dose of aerosolized anthrax spores after immunization with either pDNA-DMRIE:DOPE or pDNA-Vaxfectin™ vaccines.

While vaccination against influenza has been reported as the most cost-effective approach, development and manufacture of currently licensed influenza vaccines require the use of technologies that have proven slow and unreliable and are therefore inadequate to meet the challenges of a potentially rapidly changing and spreading pandemic. One potential technology that may meet the requirements for rapid manufacture of influenza vaccines is pDNA. But pharmaceutical grade pDNA production requires bacterial fermentation followed by lengthy purification and extensive Quality Control testing. There are also some concerns that trace amounts of antibiotics and other fermentation components may carry over after purification. Ideally, a nucleic acid vaccine could be produced using a cell-free system more akin to a small molecule synthetic process.

Citation of the documents herein is not intended as an admission that any document is pertinent prior art. All statements as to the date or representation as to the contents of the documents is based on available information and does not constitute any admission as to the correctness of the dates or contents of these documents.

### BRIEF SUMMARY OF THE DISCLOSURE

This disclosure is directed to the use of linear DNA or Linear Expression Cassettes (LECs) or linear non-expressing DNA cassettes (LCs) as opposed to covalently closed circular plasmid DNA or pDNA. Advantages provided by an LEC include production by a cell-free process and the lack of a requirement for antibiotic resistance genes, an origin of replication, and other sequences unrelated to the expression of an LEC encoded gene product. Other potential advantages of certain linear DNA production methods include use of well-defined and simple reactions, ease of purification and therefore, fast vaccine production, such as where the LEC encoded gene product is an antigen to elicit an immune response, which may be an immunotherapeutic response or a state of immunity in some embodiments of the disclosure. In another embodiment, the LEC may contain a gene encoding a therapeutic protein, such as insulin, erythropoietin, or clotting Factor IV, which when expressed in the injected tissue, can elicit a therapeutic effect such as, respectively, correct glucose metabolism in diabetics, increase red blood cell numbers in anemic subjects or accelerate blood clotting in hemophiliacs.

In a first aspect, the disclosure includes an LEC with an expression optimized DNA coding sequence, operably linked to a promoter, and terminal primer sequences at each end of the LEC for polymerase chain reaction (PCR) mediated amplification. Embodiments of the disclosure include expression optimized coding sequences which increase or improve expression of the encoded polypeptide(s). Non-limiting examples include partial or complete codon optimization relative to the host or host cell in which expression is desired and/or sequence alteration within the degeneracy of the genetic code to remove predicted secondary structure. Partial codon optimization may include alteration of one or more codons in a coding sequence where the alteration increases or improves expression of the encoded sequence. Other non-limiting examples include insertion of glycosylation sites and in vitro conjugation to polyethylene glycol (PEG). Further non-limiting examples include removal, by substitution that does not affect the encoded polypeptide(s), of a cryptic splice donor or acceptor site, a poison site, Chi structures, and/or region of high G/C or A/T content.

In some embodiments, the LEC may further include one or more components in cis that facilitate the expression of the encoded sequence. Non-limiting examples of such components include a regulatory sequence operably linked to the promoter, a polyadenylation signal, and/or one or more protective features that reduce or decrease degradation of the LEC, such as that reflected by increased or improved expression of an encoded gene product by the LEC. In some cases, the protective feature is selected from phosphorothioate-modified oligonucleotide primers that become part of the LEC and/or ligation of hairpin oligonucleotides to the ends of the linear LEC DNA molecule.

The disclosed LECs are advantageously not limited by the type or length of a coding sequence present therein. The sequence may encode an RNA molecule or a polypeptide as suitable for the intended purpose of the LEC. In some embodiments, the gene product is an RNA molecule such as an anti-sense sequence, a ribozyme, an siRNA, or a microRNA. In other embodiments, the encoded gene product beneficially codes for a polypeptide which produces an immune response when expressed in a host organism. Non-limiting examples include an LEC wherein the encoded gene product is an antigen, or fragment thereof, which produces an immune response upon expression in an animal or human subject. In further embodiments, the gene product is an endogenous (or “self”) polypeptide or RNA that is produced by the cell or organism into which the LEC is introduced. Non-limiting examples include a polypeptide or RNA with a therapeutic effect. Therefore, an LEC of the disclosure may be a therapeutic LEC.

In some cases, the immune response is protective against an organism expressing that antigen or fragment thereof. The immune response may thus be a state of immunity in some embodiments. The disclosure includes embodiments wherein the antigen is that of a pathogenic agent, such as an infectious microorganism or virus. Non-limiting examples of a pathogenic microorganism include infectious bacteria, fungi, and unicellular eukaryotes. Of course, a fragment of an antigen as disclosed herein may contain one or more epitopes that produce the described immune response.

In additional embodiments, the coding sequence of a disclosed LEC encodes a patient specific anti-idiotype product, a fusion protein comprising an antigen or fragment thereof, or a concatemer of polypeptides, such as a concatemer of antigens or fragments thereof.

Optionally, an LEC of the disclosure may encode, and thus be capable of expressing, a bivalent or polyvalent (or multivalent) gene product. The gene product thus has a valency of more than one so as to be capable of reacting or interacting with more than one binding partner in a biological setting, such as after expression in an animal or human subject. In some embodiments, the binding partner may be an antibody or fragment thereof, a cell surface receptor or molecule, an antigen or fragment thereof, or other biological substrate in or on a cell or tissue of an animal or human subject.

A bivalent or polyvalent LEC may be simply based on the encoded gene product having more than one domain or epitope that permit reaction(s) or interaction(s) with more than one binding partner molecule. A non-limiting example is an antigen of a pathogenic agent, where the antigen may have multiple domains or epitopes that interact with more than one biological molecules, such as more than one antibodies or antibodies and cell surface receptor(s) and antibodies. In other embodiments, the gene product is a fusion or chimeric polypeptide or nucleic acid molecule comprising sequences that are normally not present together in a single reading open reading frame. The sequences may be considered heterologous relative to each other.

Additionally, a bivalent or polyvalent LEC may be based on the presence, and then expression, of more than one coding region in the LEC. In other embodiments, the increased valency is based on the presence, and then expression of a coding region that encodes a gene product with more than one domain or epitope for reaction or interaction with a binding partner.

Additionally, an LEC of the disclosure may be in the form of a concatemer of more than one LEC as described herein. In some embodiments, a concatemer may be formed prior to use of an LEC to express the encoded gene product(s). In other embodiments, an LEC may be introduced into a cell or organism as monomers which become concatemers in the cell or organism. In alternative embodiments, the introduced LEC may be a mixture of monomers and concatemers.

In a second aspect, the disclosure includes a composition containing a particle, bead, polymer or suspendable solid support removably associated with an LEC as described herein. Thus in some embodiments, the LEC includes a DNA coding sequence operably linked to a promoter and terminal primer sequences for PCR mediated amplification of the LEC. In this aspect, an LEC may be associated with a particle, bead, polymer or suspendable solid support via covalent and/or non-covalent means. In some embodiments, a particle or bead may be made of, or coated with, gold and/or tungsten prior to association with an LEC. The association includes specific or non-specific conjugation of an LEC to a suspendable solid support. Additional embodiments include a solid lipid nanoparticle (SLN) or cationic SLN (see for example Pedersen et al. Eur. J. Pharm. Biopharm. 2006, 62(2):155-62); gelatin nanoparticles (see for example Zwiorek et al. J. Pharm. Pharm. Sci. 2005, 7(4):22-8); nanoparticles (see for example Prow et al. Mol. Vis. 2006, 12:606-15); and magnetic particles or beads (see for example Day et al. Biochem. J. 1991, 278(Pt. B):735-40). Further embodiments include a solid support suitable or compatible for use with a bolistic delivery system, such as a commercially available “gene gun.”

In further embodiments, an LEC may be attached to a suspendable solid support via the 5′ end of a primer used to synthesize the LEC. In some cases, the primer is that used in a PCR reaction to produce the LEC. In some embodiments, a primer is attached to the solid support prior to its use in a polymerization reaction to produce an LEC of the disclosure. The polymerization reaction may be polymerase-mediated or by de novo synthesis.

In another aspect, the disclosure includes a composition comprising a carrier associated with an LEC as disclosed herein. Non-limiting examples of a carrier include mono- or poly-cationic molecules which can complex with an LEC via ionic interactions. In some embodiments, the cationic molecule is a cationic lipid such that the carrier is a lipid vesicle or lipoplex comprising cationic lipids. In other embodiments, the cationic molecule is a polymer or polypeptide such that the carrier is a polyplex carrier comprising cationic polymers (see for example, Kodama et al. Curr Med. Chem. 2006, 13(18):2155-61).

A composition of the disclosure may comprise a carrier and an LEC that is an expression optimized LEC comprising a DNA coding sequence operably linked to a promoter and terminal primer sequences for PCR mediated amplification of the LEC. Alternative compositions comprise an LEC that is not expression optimized. In further embodiments, the composition is formulated in a unit dosage form with an excipient, adjuvant, stabilizer, and/or a pharmaceutically acceptable carrier, which may be a carrier as described herein.

A lipid vesicle carrier of the composition may contain lipids suitable for the formation of vesicles, such as, but not limited to, a cationic lipid and/or a neutral lipid. Non-limiting examples of vesicles include unilaminar vesicles, including micelles and liposomes, and multilamellar vesicles (MLVs), or combinations thereof. In some embodiments, the vesicle contains both a cationic and a neutral lipid, optionally in equimolar amounts. In some cases, the cationic lipid is VC1052 and the neutral lipid is DPyPE.

Embodiments of the disclosure include a lipid vesicle carrier containing an LEC within the lumen of the vesicle as well as a lipid vesicle carrier that has an LEC associated with the lipid layer or exterior of the carrier. Of course combinations of such carriers are also within the scope of the disclosure. The molar ratio of LEC to cationic lipid or total lipid in a composition may range from about 8:1 to about 1:8, or even higher proportion of lipid. In some embodiments, the molar ratio is about 7:1, about 6:1, about 5:1, about 4:1, about 3:1, about 2:1, about 1:1, about 1:2, about 1:3, about 1:4, about 1:5, about 1:6, or about 1:7.

An LEC or LEC containing composition of the disclosure is optionally lyophilized or vitrified, such as for purposes of storage or increasing stability. Lyophilization (freeze-drying) and vitrification (formation of a glass-like frozen solid without the formation of ice crystals) may be by any means known to the skilled person.

As a yet further aspect, the disclosure includes a method of making or amplifying an LEC by use of PCR as well as a product LEC produced thereby. The PCR may be by thermal cycling or isothermal means as known to the skilled person. Thus, in one non-limiting embodiment, the disclosure includes an LEC produced by PCR amplification of a nucleic acid template containing the sequences of an LEC as disclosed herein. In some cases, the template includes i) an expression optimized DNA coding sequence operably linked to a promoter and ii) terminal primer sequences for PCR. The choice of PCR mediated production of LECs is distinct from other reported methods for producing LECs, such as enzymatic digestion of pDNA.

In some embodiments, a method of expressing an LEC encoded gene product in an animal, human, or plant subject is disclosed. Another embodiment is a method of producing an immune response in an animal or human subject. Such methods may include administering a disclosed LEC or composition to the subject, such as by injection or microparticle delivery as non-limiting examples. In some cases, the injection is intramuscular injection into a subject.

In other embodiments, a method of treating an animal or human subject is disclosed wherein the treatment includes administration of a disclosed LEC or an LC (linear cassette with no expression) as an adjuvant in combination with an active agent used in the treatment. The LEC or LC may be further modified to increase its therapeutic effect by inserting therapeutic motifs, such as but not limited to, CpG motifs. The active agent may be a small organic molecule; a protein, polypeptide, or peptide; a non-nucleic acid based drug; or an antigen, such as a vaccine. An LEC and an active agent may be co-administered or administered sequentially in any order. Repeated administrations of one or both of the LEC and active agent is also within the scope of the disclosure. Additional delivery methods are disclosed herein.

The details of additional embodiments are set forth in the accompanying drawings and the description below. Other features, objects, and advantages of the embodiments will be apparent from the drawings and detailed description, and from the claims.

### BRIEF DESCRIPTION OF THE DRAWINGS

FIG. 1 depicts an illustrative, and non-limiting, Linear Expression Cassette of the disclosure. PCR-generated linear DNA is shown in relation to the linearized pDNA template. Expression cassette contains the Human CMV promoter and intron A, the influenza A hemagglutinin (HA) ORF and the rabbit beta globin (RBG) terminator. Kanamycin resistance (Kan^{r}) gene in the pDNA template is also indicated.

FIG. 2 illustrates in vitro characterization of LECs. Part (A) shows a representative agarose gel depicting H3HA and H1HA LECs. Lane 1 H3HA-LEC, lane 2 MOD-H3HA-LEC, lane 3 H1HA-LEC, lane 4 MOD-H1HA-LEC, lanes labeled M contain DNA ladder. Part (B) illustrates in vitro activity of LECs and MOD-LEC as tested by transfecting mouse melanoma cells with LECs followed by Western blot analysis of cell lysates (see Example 1 herein). Cross-reacting HA band is indicated with an arrow.

FIG. 3 illustrates average results from vaccination with PBS-formulated LEC. Mice (n=15) were vaccinated on Days 0 and 21 with either 50 μg of H3HA-LEC or H1HA-LEC. H3HA-pDNA (100 μg) and PBS-only groups were included as positive and negative controls, respectively. Mice were challenged with a lethal dose of mouse-adapted Influenza A/HK/8/68 (H3N2) on Day 42 as described in Example 1 herein. After challenge survival data are presented in part (A); average group weights are shown in part (B).

FIG. 4 illustrates average results from vaccination with Vaxfectin™-formulated LEC. Mice (n=10) were vaccinated on Days 0 and 21 with either PBS-formulated H3HA-LEC (50 μg) or Vaxfectin™-formulated H3HA-LEC (50 μg) or Vaxfectin™-formulated MOD-H3HA-LEC (50, 10 and 2 μg). In addition, Vaxfectin™-formulated H3HA pDNA (100 μg) and PBS groups were included as positive and negative controls, respectively. Mice were challenged with mouse-adapted Influenza A/HK/8/68 (H3N2) on Day 42 as described in Example 1 herein. After challenge survival data are presented in part (A); average group weights are shown in part (B).

FIG. 5 illustrates average results from single administration of Vaxfectin™-formulated LEC. LEC vaccine was administered at Day 0; mice (n=10) were challenged on Day 42 as described in Example 1 herein. Groups included PBS-formulated H3HA-LEC (50 μg) or Vaxfectin™-formulated H3HA-LEC (50 μg) or Vaxfectin™-formulated MOD-H3HA-LEC (50, 10 and 2 μg). In addition, Vaxfectin™-formulated H3HA pDNA (100 μg) and PBS groups were included as positive and negative controls, respectively. Survival and weight data are presented in parts (A) and (B), respectively.

### DETAILED DESCRIPTION AND MODES OF PRACTICING THE DISCLOSURE

General

An LEC as described herein may be used as an expression platform to produce an encoded gene product in a target cell or host organism. In some embodiments, an LEC is used to express a gene product as a method to induce, promote, or generate an immune response in a host animal. In other embodiments, the expressed gene product is a therapeutic polypeptide or RNA, such as siRNA, ribozyme, or antisense oligonucleotide non-limiting examples. In other embodiments, a method of treating an animal or human subject is disclosed wherein the treatment includes administration of a disclosed LEC or an LC (linear cassette with no expression) as an adjuvant in combination with an active agent used in the treatment. The LEC or LC may be further modified to increase its therapeutic effect by inserting therapeutic motifs, such as but not limited to, CpG motifs. The active agent may be a small organic molecule; a protein, polypeptide, or peptide; a non-nucleic acid based drug; or an antigen, such as a vaccine. An LEC and an active agent may be co-administered or administered sequentially in any order. Repeated administrations of one or both of the LEC and active agent is also within the scope of the disclosure. In still other embodiments the LEC itself may function as an adjuvant. In the later, a PCR-generated LEC could be engineered to express any one of a number of proteins with adjuvant activity, such as heat shock proteins, histocompatibility proteins or cytokines. Such LECs also could co-express an antigen or be co-administered with one or more additional LECs encoding antigen(s) or with non-LEC protein, carbohydrate or lipids antigens or inactivated, split or attenuated vaccines. In the latter embodiment, a PCR-generated LC DNA will itself be an adjuvant that can be co-administered with one or more additional LECs encoding antigen(s) or with non-LEC protein, carbohydrate or lipids antigens or inactivated, split or attenuated vaccines. Additional delivery methods are disclosed herein.

In some cases, an LEC of the disclosure is used as part of a rapid production of an effective vaccine or therapeutic in response to a pathogenic agent or disease condition. The adaptability to rapid production is distinct from other methods for producing a vaccine or therapeutic, which rely on slower technology and which are inadequate to meet demands such as that of an emerging disease outbreak or rapidly advancing disease. For example, and due to the ease of production by use of PCR, nucleic acid based LEC vaccine provides a promising alternative to egg-grown and cell culture-based vaccines.

Linear Expression Constructs

As disclosed herein, an LEC with an expression optimized DNA coding sequence may be used to express a gene product in a target cell, host organism, or subject as described herein. From some perspectives, an LEC may be considered a nucleic acid (DNA) “vector.” A skilled person would understand that a “vector” is a nucleic acid molecule containing a nucleic acid sequence where the molecule can be used to introduce the sequence into a cell, such as for expression of the sequence. A nucleic acid sequence present in a vector may be “exogenous” or “heterologous” to the other vector sequences. An exogenous or heterologous sequence must be inserted into, or ligated to, the vector sequences. Additionally, an exogenous or heterologous sequence may be foreign to a target cell, host organism, or subject into which the sequence is introduced via a vector. Alternatively, a nucleic acid sequence present in a vector is found within (or native to) the target cell, host organism, or subject but is still exogenous or heterologous relative to the vector sequences.

In some embodiments, an LEC is that which is integrated into the cellular genome after introduction into a target cell, such as a cell of a host organism or animal or human patient. In other embodiments, an LEC remains episomal relative to a cellular genome. In cases of an integrated LEC containing a nucleic acid sequence found in the cellular genome, the site of integration may be that of genomic (or native) copy of the sequence. Alternatively, the site of integration in the genome may be where the vector borne sequence is ordinarily not found.

Where an LEC expresses an encoded sequence in a cell, the LEC may be considered an expression vector. A skilled person would understand that an expression vector is capable of expressing a nucleic acid sequence coding for all or part of a gene product. In some cases, the gene product is one which is translated from an intermediate messenger RNA molecule to produce a protein, polypeptide, or peptide. In other cases, the gene product is an RNA molecule that is not translated. Non-limiting examples of an RNA molecule as a gene product include an anti-sense sequence, a ribozyme, an siRNA, or a microRNA. An LEC of the invention is not limited by the length of the encoded RNA sequence. Non-limiting examples include sequences encoding an RNA molecule of about 50, about 75, about 100, about 150, about 200, about 250, about 300, about 350, about 400, about 450, about 500, about 750, or about 1000 nucleotides or longer in size.

In additional embodiments, an LEC may comprise components for expression in a non-animal or non-human species. After transfection into such a species, the LEC directs production of the encoded gene product. In some cases, the transfection may be of a plant species with an LEC containing an expression optimized DNA coding sequence, operably linked to cis elements that permit expression of the coding sequence in the plant. Non-limiting examples of such cis elements include promoter, enhancer, ribozyme initiation or internal entry signal, polyadenylation signal, and termination signal, all of which are well known to the skilled person. In other cases, the transfection may be of a microorganism, such as a bacterium, yeast or fungus, or other unicellular eukaryote. In such a case, an LEC may comprise components for expression in such organisms. Alternatively, transfection may be of a cell or cell line which permits expression of the LEC encoded gene product. In some cases, the cell or cell line is from a eukaryote, mammal, primate, and/or human source.

The expression in such alternative species or cells may be used for production of the gene product for isolation and/or subsequent use. Advantageously, the gene product may be a polypeptide for use as a therapeutic, a binding reagent, an immunogenic agent, or a vaccine. In some cases, the gene product may be an antibody or an antigen binding fragment thereof. Alternatively, the gene product may be an RNA molecule as described herein.

In further embodiments, in vitro expression of an LEC may be by a cell-free system that produces the gene product for isolation and/or subsequent use as described above.

DNA Coding Sequence

An LEC of the disclosure is not limited by the DNA coding sequence present therein. In some embodiments, a DNA coding sequence in a disclosed LEC codes for an RNA which is not translated and is a therapeutic agent. Non-limiting examples include an anti-sense sequence, a ribozyme, an siRNA, or a microRNA as described herein. When expressed intracellularly, the RNA may be used to target or regulate gene expression of one or more cellular genes, such as an oncogene as a non-limiting example to treat cancer. Other conditions that may be treated include Alzheimer's Disease and macular degeneration as non-limiting examples.

In other embodiments, a DNA coding sequence in a disclosed LEC codes for a polypeptide through an intermediate messenger RNA which is translated. In such cases, the coding sequence may be considered an open reading frame (ORF) capable of being transcribed and translated to express the encoded polypeptide. An LEC of the disclosure may contain one or more coding sequence or ORFs. An ORF is a sequence in a nucleic acid molecule “read” as a series of three consecutive nucleotides (or codons), each of which code for an amino acid in accordance with the genetic code, until the presence of a termination codon (i.e., TAA, TAG, TGA as may be found within a DNA molecule. A coding sequence, or ORF of the disclosure may be that found in any organism or virus. In the case of an ORF, it may encode all or part of a peptide, polypeptide or protein of the organism or virus.

In some embodiments, a disclosed LEC contains a sequence coding for an immunogen.

An “immunogen” is meant to encompass any antigenic or immunogenic polypeptides including poly-amino acid materials having epitopes or combinations of epitopes, and immunogen-encoding polynucleotides. In addition, an “immunogen” is also meant to encompass any poly-saccharide material useful in generating immune response. As used herein, an antigenic polypeptide or an immunogenic polypeptide is a polypeptide which, when introduced into a vertebrate, reacts with the immune system molecules of the vertebrate, i.e., is antigenic, and/or induces an immune response in the vertebrate, i.e., is immunogenic. It is quite likely that an immunogenic polypeptide will also be antigenic, but an antigenic polypeptide, because of its size or conformation, may not necessarily be immunogenic. Examples of antigenic and immunogenic polypeptides include, but are not limited to, polypeptides from infectious agents such as bacteria, viruses, parasites, or fungi, allergens such as those from pet dander, tapeworms, plants, dust, and other environmental sources, as well as certain self polypeptides, for example, tumor-associated antigens.

Antigenic and immunogenic polypeptides of the present invention can be used to prevent or treat, i.e., cure, ameliorate, lessen the severity of, or prevent or reduce contagion of viral, bacterial, fungal, and parasitic infectious diseases, as well as to treat allergies.

In addition, antigenic and immunogenic polypeptides of the present invention can be used to prevent or treat, i.e., cure, ameliorate, or lessen the severity of cancer including, but not limited to, cancers of oral cavity and pharynx (i.e., tongue, mouth, pharynx), digestive system (i.e., esophagus, stomach, small intestine, colon, rectum, anus, anal canal, anorectum, liver, gallbladder, pancreas), respiratory system (i.e., larynx, lung), bones, joints, soft tissues (including heart), skin, melanoma, breast, reproductive organs (i.e., cervix, endometirum, ovary, vulva, vagina, prostate, testis, penis), urinary system (i.e., urinary bladder, kidney, ureter, and other urinary organs), eye, brain, endocrine system (i.e., thyroid and other endocrine), lymphoma (i.e., hodgkin's disease, non-hodgkin's lymphoma), multiple myeloma, leukemia (i.e., acute lymphocytic leukemia, chronic lymphocytic leukemia, acute myeloid leukemia, chronic myeloid leukemia).

Examples of viral antigenic and immunogenic polypeptides include, but are not limited to, adenovirus polypeptides, alphavirus polypeptides, calicivirus polypeptides, e.g., a calicivirus capsid antigen, coronavirus polypeptides, distemper virus polypeptides, Ebola virus polypeptides, enterovirus polypeptides, flavivirus polypeptides, hepatitis virus (AE) polypeptides, e.g., a hepatitis B core or surface antigen, herpesvirus polypeptides, e.g., a herpes simplex virus or varicella zoster virus glycoprotein, immunodeficiency virus polypeptides, e.g., the human immunodeficiency virus envelope or protease, infectious peritonitis virus polypeptides, influenza virus polypeptides, e.g., an influenza A hemagglutinin, neuramimidase, nucleoprotein, and/or matrix or M2 polypeptides, leukemia virus polypeptides, filovirus polypeptides, orthomyxovirus polypeptides, papilloma virus polypeptides, parainfluenza virus polypeptides, e.g., the hemagglutinin/neuramimidase, paramyxovirus polypeptides, parvovirus polypeptides, pestivirus polypeptides, picoma virus polypeptides, e.g., a poliovirus capsid polypeptide, pox virus polypeptides, e.g., a vaccinia virus polypeptide, rabies virus polypeptides, e.g., a rabies virus glycoprotein G, reovirus polypeptides, retrovirus polypeptides, and rotavirus polypeptides.

Examples of bacterial antigenic and immunogenic polypeptides include, but are not limited to, Actinomyces polypeptides, Bacillus polypeptides such as Anthracis bacillus PA and/or LF polypeptides, Bacteroides polypeptides, Bordetella polypeptides, Bartonella polypeptides, Borrelia polypeptides, e.g., B. burgdorferi OspA, Brucella polypeptides, Campylobacter polypeptides, Capnocytophaga polypeptides, Chlamydia polypeptides, Clostridium polypeptides, Corynebacterium polypeptides, Coxiella polypeptides, Dermatophilus polypeptides, Enterococcus polypeptides, Ehrilichia polypeptides, Escherichia polypeptides, Francisella polypeptides, Fusobacterium polypeptides, Haemobartonella polypeptides, Haemophilus polypeptides, e.g., H. influenzae type b outer membrane protein, Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides, Proteus polypeptides, Pseudomonas polypeptides, Rickettsia polypeptides, Rochalimaea polypeptides, Salmonella polypeptides, Shigella polypeptides, Staphylococcus polypeptides, Streptococcus polypeptides, e.g., S. pyogenes M proteins, Treponema polypeptides, and Yersinia polypeptides, e.g., Y. pestis F1 and V antigens.

Examples of fungal immunogenic and antigenic polypeptides include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite immunogenic and antigenic polypeptides include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides, e.g., P. falciparum circumsporozoite (PfCSP), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of helminth parasite immunogenic and antigenic polypeptides include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyrne polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides.

Examples of ectoparasite immunogenic and antigenic polypeptides include, but are not limited to, polypeptides (including protective antigens as well as allergens) from fleas; ticks, including hard ticks and soft ticks, flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

Examples of tumor-associated antigenic and immunogenic polypeptides include, but are not limited to, tumor-specific immunoglobulin variable regions, GM2, Tn, sTn, Thompson-Friedenreich antigen (TF), Globo H, Le(y), MUC1, MUC2, MUC3, MUC4, MUC5AC, MUC5B, MUC7, carcinoembryonic antigens, beta chain of human chorionic gonadotropin (hCG beta), HER2/neu, PSMA, EGFRvIII, KSA, PSA, PSCA, GP100, TRP 1, TRP 2, tyrosinase, MART-1, PAP, CEA, BAGE, MAGE1 and MAGE 2, RAGE, and related proteins.

In other embodiments, an LEC comprises a sequence encoding a “toxoid” which results in the formation of antitoxins in vivo for active immunity. Non-limiting examples include “toxoids” of Botulinum, tetanus, and diphtheria (where each of these microbes produces a corresponding toxin).

In alternative embodiments, an LEC encoded gene product is an antibody or antigen binding fragment thereof. Expression of such a gene product may be used to produce an immune response in a treated animal or human subject mediated by the antibody, or antigen binding fragment thereof. In some cases, the immune response is mediated by the binding of the antibody, or antigen binding fragment thereof, to its cognate antigen within the subject. Advantageously, the antigen may be that of a pathogenic agent as described herein and the immune response may be to the agent.

Of course, the polypeptides may be all or part of the whole polypeptide as present in the wildtype organism. Optionally, the immune response is a protective response against the organism expressing the polypeptide.

In additional embodiments, the coding sequence of a disclosed LEC encodes a patient specific anti-idiotype product, a fusion protein comprising an antigen or fragment thereof, or a concatemer of polypeptides, such as a concatemer of antigens or fragments thereof. The production of sequences encoding a fusion protein or a concatemer of antigens or fragments thereof may be by recombinant or de novo synthesis means or other means known to the skilled person. The fusion protein may be of more than one, such as 3 or more, antigens and/or toxoids such that a multivalent gene product may be produced by the LEC. Non-limiting examples include a fusion of antigenic epitopes from diphtheria, tetanus, and pertussis.

In some cases, an LEC encoded polypeptide is an immunomodulator which can increase or amplify an immune response, like an adjuvant, or decrease or suppress an immune response like an immunosuppressant. An adjuvant augments induction, promotion, or production of an immune response against an encoded antigen or fragment thereof. Non-limiting examples of an immunomodulator include a cytokine, interleukin, interferon, GM-CSF, G-CSF, M-CSF, tumor necrosis factor, chemokines, and combinations thereof. In other cases, an LEC encoded polypeptide is an angiogenic factor or an anti-angiogenic factor. Non-limiting examples include angiostatin, thrombospondin, and endostatin.

In yet additional embodiments, an LEC encoded polypeptide is a therapeutic agent. Non-limiting examples of such a polypeptide include erythropoietin (EPO), a blood clotting factor, such as factor VIII, factor IX, and insulin. Of course, the expressed polypeptide may be from the same species as the subject to be administered the LEC. Thus, a human patient may receive an LEC expressing human EPO or a human factor IX.

In further embodiments, a coding sequence further comprises a sequence encoding a secretion signal to form an ORF of the signal with the encoded polypeptide. The resultant fusion polypeptide, when expressed in a cell, is thus more likely to be secreted from the cell. The selection of secretion signals suitable for combination with a given target cell, such as that of an animal or human subject, is known to the skilled person. In some cases, the resultant polypeptide is an antigen, or fragment thereof, comprising a secretion signal.

Optionally, an LEC further comprises a sequence encoding a proteinaceous nuclear localization signal (NLS) or a nucleic acid moiety that directs delivery of the LEC to the nucleus of a cell. Non-limiting examples include one or more karyophilic proteins attached to an LEC to facilitate its localization at a nuclear pore and/or nuclear import. In additional embodiments, an LEC expressed gene product may comprise a moiety which targets the gene product to a cellular organelle, such as but not limited to, mitochondria or plastids of a cell. Non-limiting examples of a moiety include a mitochondrial localization signal (MLS) which is encoded by a sequence that is fused in-frame with the coding sequence for the gene product. The MLS may be located at the N- or C-terminus of a gene product's coding sequence.

In other embodiments, an LEC comprises a proteinaceous moiety that targets a cell type, such as via a particular cell surface receptor. Non-limiting examples include cell surface receptors found on particular immune system cells, such as subtypes of T cells and B cells.

Also included as polypeptides of the present invention are fragments or variants of the foregoing polypeptides, and any combination of the foregoing polypeptides. Additional polypeptides may be found, for example in “Foundations in Microbiology,” Talaro, et al., eds., McGraw-Hill Companies (October, 1998), Fields, et al., “Virology,” 3rd ed., Lippincott-Raven (1996), “Biochemistry and Molecular Biology of Parasites,” Man, et al., eds., Academic Press (1995), and Deacon, J., “Modern Mycology,” Blackwell Science Inc (1997).

The immunogen-encoding polynucleotide is intended to encompass a singular “polynucleotide” as well as plural “polynucleotides,” and refers to an isolated molecule or construct. The immunogen-encoding polynucleotides include nucleotide sequences, nucleic acids, nucleic acid oligomers, messenger RNA (mRNA), DNA (e.g. pDNAs, derivatives of pDNA, linear DNA), or fragments of any of thereof.

The form of immunogen-encoding polynucleotides depends in part on the desired kinetics and duration of expression. When long-term delivery of a protein encoded by a polynucleotide is desired, the preferred form is DNA. Alternatively, when short-term transgene protein delivery is desired, the preferred form is mRNA, since mRNA can be rapidly translated into polypeptide; however RNA may be degraded more quickly than DNA.

In one embodiment, the immunogen-encoding polynucleotide is RNA, e.g., messenger RNA (mRNA). Methods for introducing RNA sequences into mammalian cells is described in U.S. Pat. No. 5,580,859. A viral alpbavector, a non-infectious vector useful for administering RNA, may be used to introduce RNA into mammalian cells. Methods for the in vivo introduction of alphaviral vectors to mammalian tissues are described in Altman-Hamamdzic, S., et al., Gene Therapy 4, 815-822 (1997).

The immunogen-encoding polynucleotide, e.g., pDNA, mRNA, polynucleotide or nucleic acid oligomer can be solubilized in any of various buffers prior to mixing or complexing with the adjuvant components, e.g., cytofectins and co-lipids. Suitable buffers include phosphate buffered saline (PBS), normal saline, Tris buffer, and sodium phosphate. Insoluble polynucleotides can be solubilized in a weak acid or weak base, and then diluted to the desired volume with a buffer. The pH of the buffer may be adjusted as appropriate. In addition, a pharmaceutically acceptable additive can be used to provide an appropriate osmolarity. Such additives are within the purview of one skilled in the art.

An LEC of the invention is not limited by the length of the coding sequence. Non-limiting examples include sequences encoding polypeptides of about 10, about 25, about 50, about 75, about 100, about 150, about 200, about 250, about 300, about 350, about 400, about 450, about 500, about 750, about 1000, about 2000, about 3000, about 4000, about 5000, about 6000, about 7000, about 8000, about 9000, about 10,000 amino acids or longer in size. In other embodiments, an LEC encoded polypeptide is of a size appropriate for intracellular processing and presentation via MHC Class I or Class II molecules. Non-limiting examples include polypeptides of 9 amino acids in length, or a longer length that is processed intracellularly to be presented via MHC Class I or Class II molecules.

Sequences encoding, and sequences of, various proteins, polypeptides and peptides are known to the skilled person and are available via publicly accessible computerized databases. A desired coding sequence may be isolated or amplified followed by introduction into an LEC of the disclosure by any means known to the skilled person.

As described herein, a coding sequence may be optimized for expression via an LEC. In some embodiments, the coding sequence is optimized by genetic engineering or recombinant techniques prior to introduction into an LEC. In other embodiments, the optimization occurs within the context of an LEC. Optimization for expression refers to the alteration of one or more bases in a coding sequence to improve the expression thereof in a target cell, host organism, or subject. In some cases, the base alteration(s) change one or more codons within the degeneracy of the genetic code such that the encoded amino acid remains the same but the coding sequence is altered. Such an alteration may introduce one or more codons that are better utilized for expressing the encoded polypeptide and/or remove one or more codons that are detrimental to expression of the polypeptide.

As a non-limiting example, a sequence alteration may be used to introduce one or more codons that are more frequently or efficiently utilized, relative to other codons encoding the same amino acid, in a human cell based upon codon preference. The resulting coding sequence may be advantageously used in an LEC for expression in human cells. Alternatively, and also as a non-limiting example, a sequence alteration maybe used to remove a codon that is infrequently or inefficiently utilized in a human cell such that another codon encoding the same amino acid is introduced. The introduced codon need not be limited to the most frequently used, or most efficient, of the possible codons for use in a human cell.

In further embodiments, a base substitution may be used to remove predicted secondary structure from a coding sequence, or the LEC in which it is present, to improve expression thereof. Algorithms for the prediction and removal of secondary structure in a nucleic acid sequence are known to the skilled person and may be used as desired and/or appropriate. The removal of predicted secondary structure in a coding sequence is of course with respect to the genetic code, so as to not alter the encoded polypeptide, and with respect to the codon preference of a target cell, such as that of an animal or human subject. Additionally, an alteration to remove predicted secondary structure may be preceded by prediction of the altered sequence as not having new secondary structure introduced by the alteration.

According to the present invention, the immunogenic composition of the present invention can be used to immunize a vertebrate. The term “vertebrate” is intended to encompass a singular “vertebrate” as well as plural “vertebrates”, and comprises mammalian and avian species, as well as fish. The method for immunizing a vertebrate includes administering to the vertebrate an immunogenic composition of the present invention in an amount sufficient to generate an immune response to the immunogen.

Other non-limiting examples of expression optimization include introduction, or addition, of a glycosylation site and/or a site for in vitro conjugation to polyethylene glycol (PEG) to a disclosed LEC. Further non-limiting examples include removal, by substitution that does not affect the encoded polypeptide(s), of a cryptic splice donor or acceptor site, Chi structures, and/or region of high G/C or A/T content.

Additionally, and as described herein, a coding sequence is operatively associated or linked with a promoter, and optionally with other regulatory sequences, in an LEC. Non-limiting examples of other regulatory sequences include a trancriptional enhancer, splicing signals, a transcription termination signal, a polyadenylation signal, a translation initiation codon, a translation termination signal, and an internal ribosome entry site (IRES). Other optional regulatory sequences include those which direct an mRNA or protein gene product within a cell. The optional additional elements may be located in the 5′ and/or 3′ untranslated sequences and/or in non-transcribed sequences in some LEC embodiments.

Promoter

An LEC of the disclosure is not limited by the promoter(s) or other control sequences present therein. In some embodiments, an LEC will comprise at least one promoter, which is a nucleic acid sequence which regulates initiation, and possibly the rate, of transcription of a DNA sequence operatively linked or positioned relative to the promoter. A promoter may contain sequences for the binding of regulatory proteins and other molecules, such as RNA polymerase and transcription factors as non-limiting examples. A skilled person would understand the term “operatively linked” to refer to a correct location and/or orientation between a promoter and a nucleic acid sequence such that initiation of transcription by the promoter results in transcription of the nucleic acid sequence. So in embodiments where the nucleic acid sequence is an ORF, the promoter initiated transcription results in the production of an mRNA corresponding to the coding sequence. The mRNA may subsequently be translated to complete expression of the encoded protein.

In some embodiments, a promoter of an LEC is a recombinant or heterologous promoter relative to the coding sequence operatively linked thereto. A skilled person would understand a recombinant promoter to include a synthetic promoter that does not occur in nature and a heterologous promoter to include one that is not normally associated with the operatively linked coding sequence in its natural environment. A promoter of the disclosure may be of other coding sequences, including those isolated from a prokaryotic, viral, or eukaryotic or plant source. Alternatively, a promoter is that which is normally found with the coding sequence in its natural environment, such as a promoter present in the 5′ non-coding sequences located upstream of a coding sequence. While such a promoter may be considered “endogenous”, it may be isolated with the linked coding sequence or separately isolated and then recombinantly linked to the coding sequence.

Of course, the promoter of an LEC will be that which is suitable for expressing the operatively linked coding sequence in a target cell, such as that of an animal or human subject, cell, plant species, or microorganism as described herein. The skilled person has knowledge of, and may readily select, promoter/cell type combinations which allow for protein expression in a given cell. A promoter of the disclosure may be constitutive, tissue-specific, inducible, and/or otherwise able to direct high level expression of an operatively linked coding sequence. In some embodiments of the invention, the promoter is a cytomegalovirus (CMV) promoter capable of initiating expression in a variety of eukaryotic cells, including human cells. Other non-limiting examples of promoters include a Rous Sarcoma Virus (RSV) promoter, an actin promoter, a keratin promoter, a ubiquitin promoter, or an SV40 promoter.

In further embodiments, a promoter is one which allows tissue specific expression, such as a hypoxia-induced promoter, a tumor-specific promoter, a skeletal actin promoter, or a myosin promoter.

In alternative embodiments, the promoter is supercoiling independent, stronger than a CMV promoter on a linear template (such as in a eukaryotic cell), or weaker than a CMV promoter on a linear template (such as in a eukaryotic cell).

Of course, more than one promoter regulating more than one gene product may also be present in an LEC of the disclosure. The resultant LEC would be bi-cistronic or multi-cistronic. Non-limiting examples include the use of two or more promoters selected from the CMV, RSV, or SV40 promoters in an LEC to express more than one gene product.

Other Sequences in Cis

In additional embodiments of the disclosure, an LEC comprises one or more regulatory sequences or elements for use in conjunction with a promoter. In some cases, the regulatory sequence is an enhancer, which a skilled person would recognize as a cis-acting element involved in transcriptional activation. An enhancer may be a recombinant or heterologous enhancer, such as one not normally associated with a disclosed promoter or coding sequence in its natural environment. Such an enhancer may be a synthetic sequence or a sequence associated with another coding sequence. Enhancers for use in a disclosed LEC may be isolated from any other prokaryotic, viral, or eukaryotic source. Alternatively, an enhancer may be one normally located either downstream or upstream of a disclosed promoter or coding sequence in its natural environment.

In alternative embodiments, an additional regulatory sequence may be a reversible repressor or activator that may be used to regulate expression from an LEC. Non-limiting examples include a Tet responsive element, an ecdysone response element, an antiprogestin-inducible element, an oxygen level responsive element, or an antibiotic responsive element. In some cases, a hypoxia-inducible promoter may be used in an LEC suitable for transforming cells, such as some tumor cells, that are under hypoxic conditions.

A disclosed LEC may be used for expression as described herein without the presence of a termination signal to end transcription. In such embodiments, transcription may be considered “run-off” such that an RNA polymerase continues transcription until it reaches the end of the LEC. In other embodiments, an LEC comprises one or more termination signal and/or a polyadenylation signal.

A termination signal or terminator is a DNA sequence directing termination of an RNA transcript by an RNA polymerase. The presence of a termination signal ends production of an RNA transcript. In eukaryotic cells, a termination signal or region of sequence may also contain sequences that expose a polyadenylation site on the transcript to allow the addition of a polyadenylate (polyA) tail. This results in the production of polyadenylated mRNA, which may be more stable and more efficiently transferred from a cell's nucleus to the cytoplasm or more efficiently translated in a eukaryotic cell. Therefore, and in some embodiments involving eukaryotic cells, such as human cells, an LEC of the disclosure includes a polyadenylation signal. Non-limiting examples of a termination signal and/or a polyadenylation signal include that of bovine growth hormone (BGH) or Simian Virus 40 (SV40).

An additional non-limiting examples of sequences that may be present in cis on an LEC include a non-coding sequence between the promoter and the coding sequence, such as a 5′ untranslated region, and an intron sequence within a coding sequence. In the cases of the latter, the LEC is one which is for use in a cell, such as a eukaryotic cell, which correctly removes the intron prior translation. Moreover, appropriate donor and/or acceptor splicing sites are introduced as needed to ensure proper excision of introns and post-transcriptional processing for expression of an encoded polypeptide.

A disclosed LEC may further comprise an initiation of translation signal and/or an internal ribosome binding site (IRES). In some embodiments, an LEC includes an ATG initiation codon that is “in-frame” with the ORF of a coding sequence as described herein to facilitate translation of an encoded polypeptide. In other embodiments, an IRES is present in an LEC to allow expression of more than one ORF from a single transcript expressed from an LEC's coding sequence. Thus, a polycistronic LEC is within the scope of the disclosure. IRES elements able to begin translation at an internal site of an mRNA molecule have been reported. Non-limiting examples of IRES elements include those from members of the picornavirus family, namely polio and encephalomyocarditis, and a mammalian IRES element. Because an IRES element can be linked to a heterologous ORF, more than one ORFs can be expressed as a single transcript, but each separated by an IRES element, and then used as a polycistronic message to express the polypeptides encoded by the ORFs. Thus more than one ORF can be efficiently expressed using a single promoter. See, for example, U.S. Pat. Nos. 5,925,565 and 5,935,819.

In further embodiments, an LEC may comprise one or more additional control sequences, such as those beyond the elements mentioned above, and which participate in regulating or directing transcription and possibly translation of an operably linked coding sequence in a particular host organism. While a promoter and a terminator may be considered minimal control sequences, other control sequences that govern transcription and/or translation may be used.

Terminal Primer Sequences

An LEC of the disclosure further comprises terminal primer sequences at each end of the LEC molecule for PCR-mediated amplification or production of the LEC molecule. In some embodiments, the terminal primer sequences may be those present, or derived therefrom, at the ends of an LEC DNA molecule. Derived terminal primer sequences include those which contain one or more base insertions, deletions, or substitutions relative to the sequences present at the ends of an LEC. The production of a derived terminal primer sequence may be by any recombinant or de novo synthesis means known to the skilled person, including those used to alter codons as described herein. In some cases, the derivation of a terminal primer sequence is made in combination with consideration of the other sequences present in the LEC and consideration of removing predicted secondary structure which would inhibit PCR mediated replication. In other cases, the derivation is made with consideration of avoiding the generation of secondary structure in the resultant LEC molecule.

In other embodiments, the terminal primer sequences are those introduced at the ends of an LEC molecule to facilitate its replication by PCR. An introduced sequence may be selected by a skilled person based upon their knowledge and the sequence of the rest of the LEC. Moreover, and like the case with a derived terminal sequence, the selection of terminal primer sequences is made with consideration of avoiding the generation of secondary structure in the resultant LEC molecule.

LEC embodiments of the disclosure include those with a terminal primer sequence of any suitable length. Non-limiting examples include primer sequences of about 10, about 12, about 14, about 16, about 18, about 20, about 22, about 24, about 26, about 28, about 30, or about 32 bases or longer in length. Of course, the primer sequences of the two ends of an LEC need not be identical in sequence and need not be identical in length.

Protective Features

As described herein, a disclosed LEC may further include one or more components in cis that reduce or decrease degradation of the LEC such as that which may occur within an animal or human subject or within a cell. In some embodiments, the protective feature is introduced as a moiety at the 5′ or 3′ end of one or both strands of an LEC to resists exonuclease degradation. In one embodiment, the moiety is a length of noncoding DNA sequence extending beyond the promoter or terminator. In another embodiment, the moiety comprises phosphorothioate-modified oligonucleotides introduced into an LEC, such as by de novo synthesis or via primers used to replicate the LEC. Both of these moieties will result in a resistance of the DNA to degradation by exonucleases. Non-limiting examples include the use of noncoding, nonphosphorothioated oligonucleotide sequences or oligonucleotide primers where the two 5′-most residues were phosphorothioates.

In other embodiments, the moiety at the 5′ end may be part of a hairpin oligonucleotide ligated between the strands at each end of a linear LEC DNA molecule.

Alternatively, an LEC may comprise one or more phosphorothioate linkages, or bridges, in the DNA backbone.

In further embodiments, the moiety at the 5′ end is a region of peptide nucleic acid (PNA) sequence, optionally further comprising a “PNA clamp”, to protect the ends of an LEC. The PNA sequences may be introduced via the PCR primers used to replicate an LEC. Non-limiting examples include PCR primers that are wholly or partly PNA in nature. An LEC replicated by such primers comprise a PNA tail at each of the 5′-ends. The 3′ ends may be optionally protected via addition of a PNA clamp.

Molecules called PNA “clamps” have been synthesized which have two identical PNA sequences joined by a flexible hairpin linker containing three 8-amino-3,6-dioxaoctanoic acid units. When a PNA clamp is mixed with a complementary homopurine or homopyrimidine DNA target sequence, a PNA-DNA-PNA triplex hybrid can form which has been shown to be extremely stable (Bentin et al., Biochemistry 35:8863-8869, 1996; Egholm et al., Nucleic Acids Res. 23:217-222, 1995; Griffith et al., J. Am. Chem. Soc. 117:831-832, 1995).

Compositions and Formulations

The disclosure includes compositions and formulations comprising an LEC as describe herein. In some embodiments, a composition is disclosed wherein an LEC is removably associated with a suspendable solid support. Non-limiting examples include a particle, bead, or polymer which is removably associated with an LEC, such as to facilitate its handling or transfer properties. The association may be by covalent and/or non-covalent means. In cases of association mediated by a covalent bond, the bond may be one which is cleaved by an enzymatic activity, such as an enzyme that is present in a target cell, or an animal or human subject, to which the composition is delivered. The delivery may be by microprojectile bombardment with the composition.

In cases of association mediated by non-covalent interactions, the association may be that which is sufficiently stable to remain until after delivery of the composition to a target cell, such as that in an animal or human subject. Thus, in some embodiments, the composition is sufficiently stable to resist separation of the LEC and the solid support until after introduction into the target cell. In other embodiments, the composition allows separation of the LEC and the solid support after delivery to an animal or human subject such that the LEC may be taken up by a target cell.

The number of copies of LEC on each solid support may vary as permitted by the chemistries of the solid support and the means of association. In some embodiments, more than one LEC molecule is associated with each solid support. So embodiments of the invention include about 2, about 4, about 6, about 8, about 10, about 12, about 14, about 16, about 18, about 20 or more copies of an LEC with each particle, bead, polymer or other solid support medium.

In some embodiments, a suspendable solid support is attached to an LEC via a 5′ end of a primer used to replicate the LEC. A non-limiting example is a PCR primer used to produce the LEC. The attachment may occur between the solid support and the LEC after its production or be between the solid support and the primer, which is subsequently used to synthesize the LEC, which would thus be attached to the solid support. In the latter case, and where the synthesis is via PCR, only one of the two PCR primers need be associated with the solid support.

In alternative embodiments, a suspendable solid support is attached to a 3′ end of an LEC. Non-limiting examples include use of a disulfide thiol modification to introduce a 3′-thiol linkage, a 3′-amine modifier to introduce a primary amine moiety, and the addition of a 3′-phosphate group to the 3′ end.

Formulations

According to the present invention, the immunogen-encoding polynucleotides can be complexed with adjuvant compositions by any means known in the art, e.g., by mixing a pDNA solution and a solution of cytofectin/co-lipid liposomes. In one embodiment, the concentration of each of the constituent solutions is adjusted prior to mixing such that the desired final pDNA/cytofectin:co-lipid ratio and the desired pDNA final concentration will be obtained upon mixing the two solutions. For example, if the desired final solution is to be physiological saline (0.9% weight/volume), both pDNA and cytofectin:co-lipid liposomes are prepared in 0.9% saline and then simply mixed to produce the desired complex. The cytofectin:co-lipid liposomes can be prepared by any means known in the art. For example, one can hydrate a thin film of GAP-DMORIE and co-lipid mixture in an appropriate volume of aqueous solvent by vortex mixing at ambient temperatures for about 1 minute. Preparation of a thin film of cytofectin and co-lipid mixture is known to a skilled artisan and can be prepared by any suitable techniques. For example, one can mix chloroform solutions of the individual components to generate an equimolar solute ratio and subsequently aliquot a desired volume of the solutions into a suitable container where the solvent can be removed by evaporation, e.g., first with a stream of dry, inert gas such as argon and then by high vacuum treatment.

The disclosure further includes a composition comprising a lipid vesicle carrier associated with a disclosed LEC. In some cases, a vesicle is unilamellar or multilamellar. Thus micelles, liposomes, and multilamellar vesicles, or combinations thereof may be used to enclose an LEC. In other embodiments, LEC molecules may be associated with the lipid layer or exterior of a vesicle. As a non-limiting, representative example, an LEC of the disclosure may be contained within a liposome, which is composed of a phospholipid bilayer membrane enclosing an inner space or lumen, which is commonly aqueous in character. Alternatively, an LEC may be present in the bilayer, or on the exterior, of the liposome. Of course, LEC molecules may be present in more than one of these possible liposomal locations.

A multilamellar vesicle, or liposome, has multiple lipid layers separated by medium contained within intra-layer space, which is also commonly aqueous in character. In some embodiments, an LEC is contained within the intra-layer space of a multilamellar vesicle. In some embodiments, an LEC is complexed with Lipofectamine (Invitrogen).

Alternatively, an LEC is complexed with a cationic polymeric dendrimer, such as Superfect (Qiagen).

In further embodiments of the disclosure, a vesicle may be complexed with a hemagglutinating virus (HVJ), which has been shown to facilitate fusion with a cell membrane. This would promote cell entry of a liposome-encapsulated LEC.

Additionally, the disclosure includes formulations of an LEC in a unit dosage form for inducing, promoting, or producing an immune response, such as a protective response. In some embodiments, the formulation comprises a pharmaceutically acceptable carrier or excipient, optionally with an adjuvant. A unit dosage is that which is sufficient and/or effective to induce, promote, or produce an immune response in a treated animal or human subject. The dosage may vary depending on a variety of factors, including, as non-limiting examples, the promoter used in an LEC, the characteristics of the coding sequence, the method of delivery, and the weight and type of the treated subject. Nonetheless, the unit dosage form for an LEC and a subject may be readily determined by limited routine and repetitive study as known to the skilled person.

Non-limiting examples of amounts include about 1 nanogram to about 5 milligram, although about 10 ng, about 20 ng, about 30 ng, about 40 ng, about 50 ng, about 75 ng, about 100 ng, about 125 ng, about 150 ng, about 200 ng, about 250 ng, about 300 ng, about 350 ng, about 400 ng, about 450 ng, about 500 ng, about 550 ng, about 600 ng, about 650 ng, about 700 ng, about 750 ng, about 800 ng, about 850 ng, about 900 ng, about 900 ng, about 950 ng, about 1 μg, about 5 μg, about 10 μg, about 20 μg, about 30 μg, about 40 μg, about 50 μg, about 75 μg, about 100 μg, about 125 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 900 μg, about 950 μg, about 1 mg, about 2 mg, about 3 mg, about 4 mg, or about 5 mg or more of an LEC may be delivered in the practice of the invention.

Administration

The immunogenic compositions of the present invention may be administered according to any of various methods known in the art. For example, U.S. Pat. No. 5,676,954 reports on the injection of genetic material, complexed with cationic lipid carriers, into mice. Also, U.S. Pat. Nos. 5,589,466, 5,693,622, 5,580,859, 5,703,055, and PCT international patent application PCT/US94/06069 (WO 94/29469) provide methods for delivering DNA-cationic lipid complexes to mammals.

Specifically, the immunogenic compositions of the present invention may be administered to any tissue of a vertebrate, including, but not limited to, muscle, skin, brain, lung, liver, spleen, bone marrow, thymus, heart, lymph, blood, bone, cartilage, mucosal tissue, pancreas, kidney, gall bladder, stomach, intestine, testis, ovary, uterus, vaginal tissue, rectum, nervous system, eye, gland, tongue and connective tissue. Preferably, the compositions are administered to skeletal muscle. The immunogenic compositions of the invention also may be administered to a body cavity, including, but not limited to, the lung, mouth, nasal cavity, stomach, peritoneum, intestine, heart chamber, vein, artery, capillary, lymphatic, uterus, vagina, rectum, and ocular cavity.

Preferably, the immunogenic compositions of the present invention are administered by intramuscular (i.m.) or subcutaneous (s.c.) routes. Other suitable routes of administration include transdermal, intranasal, inhalation, intratracheal, transmucosal (i.e., across a mucous membrane), intra-cavity (e.g., oral, vaginal, or rectal), intraocular, vaginal, rectal, intraperitoneal, intraintestinal and intravenous (i.v.) administration.

Any mode of administration can be used so long as the administration results in the desired immune response. Administration means of the present invention include, but not limited to, needle injection, catheter infusion, biolistic injectors, particle accelerators (i.e., “gene guns” or pneumatic “needleless” injectors—for example, Med-E-Jet (Vahlsing, H., et al., J. Immunol. Methods 171, 11-22 (1994)), Pigjet (Schrijver, R., et al., Vaccine 15, 1908-1916 (1997)), Biojector (Davis, H., et al., Vaccine 12, 1503-1509 (1994); Gramzinski, R., et al., Mol. Med. 4, 109-118 (1998)), AdvantaJet, Medijector, gelfoam sponge depots, other commercially available depot materials (e.g., hydrojels), osmotic pumps (e.g., Alza minipumps), oral or suppositorial solid (tablet or pill) pharmaceutical formulations, topical skin creams, and decanting, use of polynucleotide coated suture (Qin et al., Life Sciences 65, 2193-2203 (1999)) or topical applications during surgery. The preferred modes of administration are intramuscular needle-based injection and intranasal application as an aqueous solution.

Determining an effective amount of an immunogenic composition depends upon a number of factors including, for example, the chemical structure and biological activity of the substance, the age and weight of the subject, and the route of administration. The precise amount, number of doses, and timing of doses can be readily determined by those skilled in the art.

In certain embodiments, the immunogenic composition is administered as a pharmaceutical composition. Such a pharmaceutical composition can be formulated according to known methods, whereby the substance to be delivered is combined with a pharmaceutically acceptable carrier vehicle. Suitable vehicles and their preparation are described, for example, in Remington's Pharmaceutical Sciences, 16.sup.th Edition, A. Osol, ed., Mack Publishing Co., Easton, Pa. (1980), and Remington's Pharmaceutical Sciences, 19.sup.th Edition, A. R. Gennaro, ed., Mack Publishing Co., Easton, Pa. (1995). The pharmaceutical composition can be formulated as an emulsion, gel, solution, suspension, lyophilized form, or any other form known in the art. In addition, the pharmaceutical composition can also contain pharmaceutically acceptable additives including, for example, diluents, binders, stabilizers, and preservatives. Administration of pharmaceutically acceptable salts of the polynucleotide constructs described herein is preferred. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like.

For aqueous pharmaceutical compositions used in vivo, use of sterile pyrogen-free water is preferred. Such formulations will contain an effective amount of the immunogenic composition together with a suitable amount of vehicle in order to prepare pharmaceutically acceptable compositions suitable for administration to a vertebrate.

Kits

The present invention also provides kits for use in delivering a polypeptide to a vertebrate. Each kit includes a container holding 1 ng to 30 mg of an immunogen-encoding polynucleotide which operably encodes an immunogen within vertebrate cells in vivo. Furthermore, each kit includes, in the same or in a different container, an adjuvant such as a composition comprising GAP-DMORIE and a co-lipid. Any of components of the pharmaceutical kits can be provided in a single container or in multiple containers. Preferably, the kit includes from about 1 ng to about 30 mg of an immunogen-encoding polynucleotide; more preferably, the kit includes from about 100 ng to about 10 mg of a immunogen-encoding polynucleotide.

Any suitable container or containers may be used with pharmaceutical kits. Examples of containers include, but are not limited to, glass containers, plastic containers, or strips of plastic or paper.

Each of the pharmaceutical kits may further comprise an administration means. Means for administration include, but are not limited to syringes and needles, catheters, biolistic injectors, particle accelerators, i.e., “gene guns,” pneumatic “needleless” injectors, gelfoam sponge depots, other commercially available depot materials, e.g., hydrojels, osmotic pumps, and decanting or topical applications during surgery. Each of the pharmaceutical kits may further comprise sutures, e.g., coated with the immunogenic composition (Qin et al., Life Sciences (1999) 65:2193-2203).

The kit can further comprise an instruction sheet for administration of the composition to a vertebrate. The polynucleotide components of the pharmaceutical composition are preferably provided as a liquid solution or they may be provided in lyophilized form as a dried powder or a cake. If the polynucleotide is provided in lyophilized form, the dried powder or cake also may include any salts, entry enhancing agents, transfection facilitating agents, and additives of the pharmaceutical composition in dried form. Such a kit may further comprise a container with an exact amount of sterile pyrogen-free water, for precise reconstitution of the lyophilized components of the pharmaceutical composition.

The container in which the pharmaceutical composition is packaged prior to use can comprise a hermetically sealed container enclosing an amount of the lyophilized formulation or a solution containing the formulation suitable for a pharmaceutically effective dose thereof, or multiples of an effective dose. The pharmaceutical composition is packaged in a sterile container, and the hermetically sealed container is designed to preserve sterility of the pharmaceutical formulation until use. Optionally, the container can be associated with administration means and/or instruction for use.

LEC Replication

The disclosure includes a method of replicating or manufacturing an LEC by use of PCR. Of course an LEC produced by the PCR mediated methods disclosed herein is also within the scope of the disclosure. In some embodiments, a replication method comprises i) denaturing a double stranded DNA template containing an LEC sequence of the disclosure, ii) contacting said denatured DNA template strands with a pair of PCR primers complementary to the terminal sequences defining the ends of the LEC and allowing said primers to anneal to said strands, and iii) contacting the annealed primers with a DNA polymerase under conditions for the synthesis of double stranded DNA. In alternative embodiments, a single stranded template may be used, such as in cases to remover the need for template denaturing.

As exemplified in the Examples section below, the disclosed PCR conditions are sufficient to produce enough DNA for in vivo animal studies as well as treatment of animal, human, and plant subjects as described herein. In addition to yield, a PCR-based method of the disclosure may comprise DNA produced with very few mutations. As a non-limiting example, a highly processive thermostable DNA polymerase with proof-reading functionality, such as Phusion™, may be used. The Phusion™ polymerase has been reported to have an error rate of about 4.4×10⁻⁷ or about 50-fold lower than that reported for Taq polymerase.

Additionally, and in some embodiments, a relatively high level of input nucleic acid template for LEC production may be used to reduce the chance of a mutation occurring in an early PCR cycle (e.g. a “jackpot” mutation) which results in production of a significant proportion of the mutated sequence as the final product. As a non-limiting example, about 5 ng of template for an LEC may be used.

Methods of Use

The disclosure further includes a method of using a disclosed LEC to induce, promote, or generate an immune response in an animal or human subject or a plant organism. The method comprises administering a disclosed LEC, or LEC containing composition or formulation, to the subject. Non-limiting examples of administering include administration by intramuscular, subcutaneous, intramedullary, intravenous, intraperitoneal, intranasal, intraocular, intrathecal, intraventricular, intravesicular, or subarachnoid injection. Additional routes of administration include oral, buccal, sublingual, rectal, transdermal or intradermal, vaginal, transmucosal or mucosal, nasal, intestinal, or parenteral delivery. Administration to particular organs, such as the brain or a bladder of an animal or human subject, may also be used.

In other embodiments, administration may be by microprojectile bombardment (MB) of an LEC or LEC containing composition. Methods comprising MB frequently include the use of a bolistic (or “gene gun”) device as known to the skilled person, and successful introduction of nucleic acids have been repeatedly reported (see for example, U.S. Pat. Nos. 5,538,880; 5,550,318; and 5,610,042; as well as WO 94/09699). In some embodiments, LECs are introduced into skin cells via MB. In further embodiments, administration may be by transdermal patches, needle arrays, needle-free injection, needle-free devices, and topical application. In other embodiments, hydrodynamic delivery of an LEC may be used.

In some embodiments, particles or beads as described herein are coated with an LEC and then delivered into target cells, such as those of an animal, human, or plant subject, by a propelling force. Non-limiting examples of materials used in, or on, a particle or bead include tungsten, platinum, or gold. While a particle or bead may be coated with an LEC by DNA precipitation onto metal, such coating is optional because a particle or bead may contain an LEC rather than be coated with it.

In further embodiments, the administration may be in combination with one or more adjuvants as known in the field of immunization protocols. Because the effects of an adjuvant is not antigen-specific, a variety of adjuvants may be used in the disclosed methods.

Non-limiting examples of adjuvants include alum; a bacterial molecule, such as muramyl dipeptide (N-acetylmuramyl-L-alanyl-D-isoglutamine or MDP) or a derivative thereof (such as the amino acid derivative threonyl-MDP or the fatty acid derivative MTPPE), or a peptidoglycan; an alkyl lysophosphilipid (ALP); BCG (bacillus Calmette-Guerin, an attenuated strain of Mycobacterium) or BCG-cell wall skeleton (CWS); a teichoic acid from Gram negative cells, such as a lipoteichoic acid (LTA), a ribitol teichoic acid (RTA), and a glycerol teichoic acid (GTA); a hemocyanins or hemoerythrin, such as keyhole limpet hemocyanin (KLH), an arthropod hemocyanin, or an arthropod hemoerythrin; a polysaccharide adjuvant, such as a pneumococcal polysaccharide, chitin, chitosan, or deacetylated chitin; trehalose dimycolate; an amphipathic and surface active agent, such as saponin or a derivative thereof like QS21 (Cambridge Biotech); Quil A; lentinen; MF-59 (or MF59); MPLA; or a detoxified endotoxin, such as that disclosed in U.S. Pat. No. 4,866,034. An additional non-limiting example is Ribi's Adjuvant System (RAS). Additional non-limiting examples of adjuvants are described by Vogel F R, et al. (“A compendium of vaccine adjuvants and excipients.” Pharm Biotechnol. 1995, 6:141-228), which is incorporated herein by reference as if fully set forth. The disclosed compositions and methods may include use of any one or more of the adjuvants described therein.

The methods of the disclosure optionally comprise multiple administrations of an LEC or composition thereof. In some embodiments, the number of repeat administrations is less than six, less than four, or about one, two, or three. The administrations may be spaced by various time intervals. Non-limiting examples include an interval from about two to about twelve weeks, about four to about six weeks, or about eight to about ten weeks. Booster administrations after an interval of about 1, about 2, about 3, about 4, or about 5 or more years may also be used.

The course of the induced, promoted, or generated immune response may be followed by assays for antibodies or immune cells against the polypeptide expressed by the administered LEC. The assays may be performed by use of an antibody or cell containing fluid from a treated subject. Non-limiting examples include serum, plasma, or blood from a subject. Representative assay techniques are disclosed in U.S. Pat. Nos. 3,791,932; 3,949,064 and 4,174,384. Assays for other immune responses can also be performed. In some embodiments, the assay is for protection from challenge with a pathogen expressing the polypeptide.

Having now generally described the invention, the same will be more readily understood through reference to the following examples which are provided by way of illustration, and are not intended to be limiting of the disclosed invention, unless specified.

### EXAMPLES

### Example 1

### Materials and Methods

PCR Amplification of LEC DNA

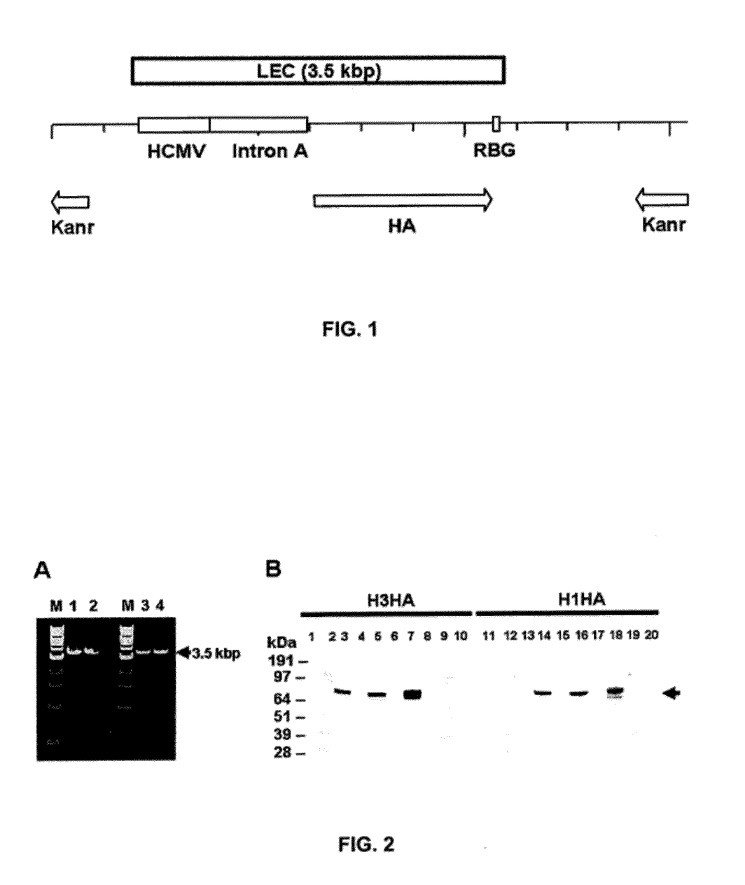

Oligonucleotide primers (forward 5′TGGCCATTGCATACGTTGTATCCATATCAT and reverse 5′ AGTCAGTGAGCGAGGAAGCGGAAGAGTACC) were designed flanking the CMV promoter and the Rabbit Beta Globin (RBG) transcription terminator of Vical's HA expression vector such that amplification produces a 3.5 kbp Linear Expression Cassette (LEC) containing CMV promoter/intron and termination sequences in addition to either the influenza A/HK/8/68 H3N2 (H3HA) or the influenza A/PR/8/34 H1N1 (H1HA) ORF (FIG. 1). Two sets of primers were used (Sigma-Genosys; Saint Louis, Mo.); the first set was made to contain only standard deoxyribonucleotides while the second set was prepared such that the two 5′-most residues in each primer were derivatized with phosphorothioate (MOD). PCR was carried out using 5 ng of linearized influenza A HA plasmid, 200 μM of each dNTP (Invitrogen; Carlsbad, Calif.), 2 U of Phusion™ and 1× Phusion™ HF buffer (Finnzymes; Espoo, Finland) in a final volume of 100 PCR was performed in an Applied Biosystems 9700 thermocycler (Foster City, Calif.) using the following temperature cycling conditions: (a) Un-modified oligonucleotides: 1×98° C. (30 s); 30×{98° C. (15 s), 50° C. (15 s), 72° C. (90 s)}; 1×72° C. 600 s and (b) MOD oligonucleotides: 1×98° C. (30 s); 35×{98° C. (15 s), 71° C. (15 s), 72° C. (90 s)}; 1×72° C. 600 s. H3HA and H1HA ORFs were cloned at Vical directly from mouse-adapted A/HK/68 H3N2 and A/PR/34 H1N1 strains, respectively.

Purification of Amplification Products

PCR amplification products were purified using DNA-binding resin columns (Qiagen; Valencia, Calif.) and concentrated using ultrafiltration on Centriplus® columns (Millipore; Bedford, Mass.). LEC DNA concentration was determined by A₂₆₀ absorption spectrophotometry using at least two dilutions. LEC DNA from several PCR runs was pooled before further testing.

LEC DNA Characterization

LEC DNA was run on TAE-EtBr 1% agarose gels to confirm the presence of the expected 3.5 kbp band and the absence of unincorporated primers and extraneous DNA bands (FIG. 2). DNA lengths were estimated by comparison to 1 kbp DNA ladder (New England Biolabs; Ipswich, Mass.). LEC preparations were further characterized by DNA sequence before in vivo studies. DNA sequence information was obtained using standard fluorescence-labeled double-stranded dideoxy sequencing technology (Retrogen; San Diego, Calif.). Sequencing was performed using primers at approximately every 500 bp on both strands. Sequence was assembled using Sequencher™ (Ver 4.1.4, Gene Codes Corporation; Ann Arbor, Mich.).

In Vitro Antigen Expression Test

A Western blot assay was used to confirm that influenza virus H1HA and H3HA proteins of the correct immunological specificity and size were expressed from the LECs. Proteins were identified by transfecting mouse melanoma cells with LECs, analyzing the cell lysates by Western blot, and verifying that the immunologically-specific protein bands were the expected apparent molecular weight. LECs were introduced into cells at a dose of 1.5 μg/mL complexed with 5 μL/mL ExGen 500 (Fermentas Life Sciences; Burlington, Canada). For positive and negative controls, plasmid with or without the transgene-specific ORF were introduced into cells at a dose of 3 μg/mL complexed with 10 μL/mL ExGen 500. At 48 hours post-transfection, the cells were harvested using Versene (Invitrogen; Carlsbad, Calif.) and lysed with lysis buffer containing 20 mM Tris-HCl, pH 8.0, 300 mM NaCl, 8 mM EDTA, pH 8.0, 10% glycerin, 0.5% NP-40, and one protease inhibitor tablet (Roche; Alameda, Calif.). Cell lysates were stored at −70° C. prior to Western blot analysis. Samples were analyzed under reducing/denaturing gel electrophoresis conditions in 4-12% Bis-Tris NuPAGE® gels (Invitrogen; Carlsbad, Calif.). The proteins were transferred to 0.2 μm PVDF membranes (Invitrogen; Carlsbad, Calif.) and probed with primary antibodies (rabbit vaccination; 2.6.1) followed by alkaline-phosphatase conjugated secondary antibodies (Jackson ImmunoResearch; WestGrove, Pa.). NBT/BCIP substrate (KPL; Gaithersburg, Md.) was used to produce visible protein bands.

Formulation

Vaxfectin™ consists of an equimolar mixture of VC1052 ((±)-N-(3-aminopropyl)-N,N-dimethyl-2,3-bis(myristoleyloxy)-1-propanaminium bromide) and DPyPE (Diphytanoylphosphatidyl-ethanolamine). Preparation of Vaxfectin™ and its formulations were first described by Hartikka and co-workers (“Vaxfectin enhances the humoral immune response to plasmid DNA-encoded antigens.” Vaccine 2001, 19(15-16), 1911-1923). A modified protocol is briefly summarized as follows. Both VC1052 and DPyPE are resuspended in chloroform, mixed in 1:1 molar ratio, aliquoted into vials, and dried to create Vaxfectin™ reagent dry lipid film. On the day of injection, the lipid film vials are resuspended in 1 mL 0.9% saline and diluted if necessary. LECs and pDNA were prepared in 0.9% saline, 20 mM sodium phosphate, pH 7.2. LECs are formulated with Vaxfectin™ by gently streaming the lipid into LECs of equal volume. All the required doses were prepared by formulating at 0.2 to 0.5 mg/mL range and diluting down to lower concentration as required.

Vaccination of Rabbits with H3HA and H1HA-Expressing Plasmids

Antibody reagents for HA LEC characterization were obtained through rabbit vaccination. For this purpose, two New Zealand White rabbits (Harlan Sprague-Dawley; Oxford, Mich.) were vaccinated with Vaxfectin™-formulated H3HA pDNA. In addition, two rabbits were vaccinated with the Vaxfectin™-formulated H1HA pDNA. One milligram of pDNA was administered intramuscularly (rectus femoris) at 1 mg/mL on Days 0, 28 and 56; final bleed was done at Day 78 by ear vein puncture.

Immunization of BALB/c Mice and Viral Challenge

The Influenza A challenge model used a mouse-adapted A/HK/8/68 strain as first described by Ulmer and co-workers (Ulmer et al.). The mouse-adapted virus causes a lethal infection in the majority of mice infected intranasally under ketamine anesthesia at a dilution of 1:10,000 of the viral stock (50 pfu). At this dose, most of the mice are dead by day 10 post-challenge. Typically, no more than 20% of naïve mice survive infection with this viral challenge dose. Female BALB/c mice six to eight weeks old (Jackson Laboratories; Bar Harbor, Minn.) were used for the influenza viral challenge studies. DNA vaccinations were given as bilateral, rectus femoris injections followed by intranasal viral challenge at three to six weeks after vaccination with 50 pfu (1 LD₉₀) of virus. Mice were monitored for morbidity and weight loss for three weeks following viral challenge. Average weight loss was not calculated for groups that lost 30% or more of the animals.

Statistical Analysis

Mouse weights were analyzed using the Mann-Whitney nonparametric statistical test. Mouse survival was analyzed using a Kaplan-Meier survival plot followed by a log-rank (Mantel-Cox) test. Differences were considered statistically significant when p≦0.05.

### Example 2

### PCR-Amplified LECs can Protect Against Influenza a Lethal Viral Challenge

A LEC-based influenza A vaccine was studied in mice using a lethal dose of a mouse-adapted virus. The study was designed to test superiority of homotypic H3HA-LEC vaccine versus heterotypic vaccine H1HA-LEC. Four groups of mice were vaccinated at day 0 and 21 and challenged at Day 42 with H3N2 influenza A/HK/8/68 mouse-adapted virus. The groups were: (1) H3HA-LEC vaccine test (50 μg/dose; 15 mice), (2) H1HA-LEC vaccine comparator (50 μg/dose; 15 mice), (3) VR4750 H3HA-pDNA (100 μg/dose; positive control, 10 mice) and (4) PBS, no DNA (15 mice; negative control-vehicle only). The primary study endpoint was survival and the secondary endpoint was weight.

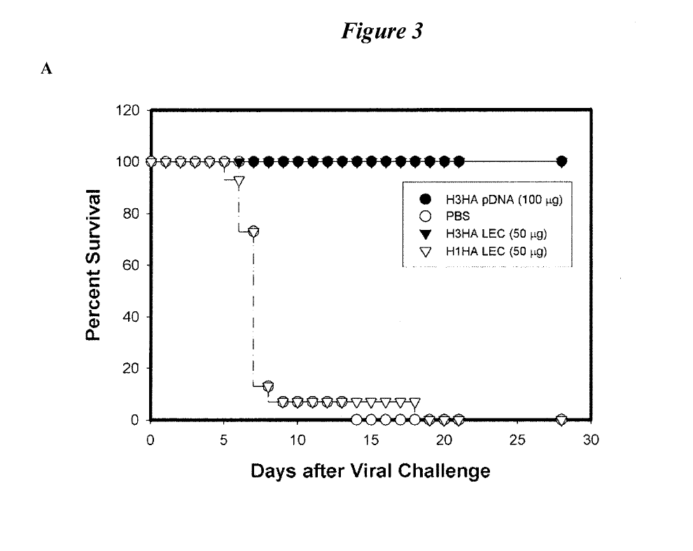

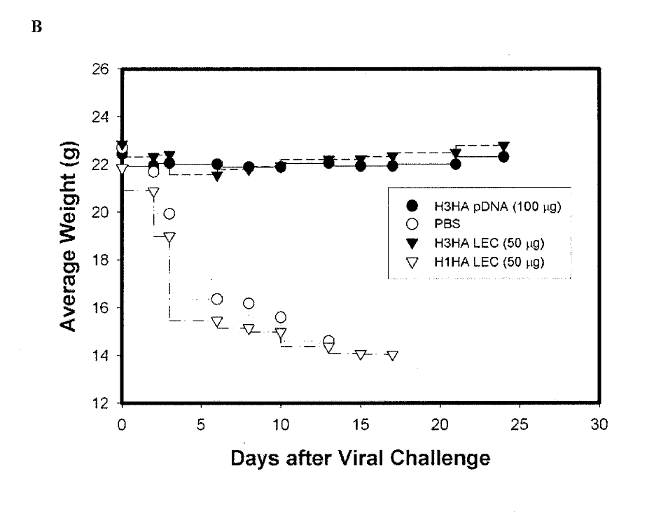

With 15 mice per test group, this study was 80% powered to test superiority between H3HA-LEC group versus H1HA-LEC group. Data from this study are summarized in FIG. 3. Mice in both the H3HA-LEC and H3HA-pDNA groups survived the lethal virus challenge, did not lose weight or appear ill. In contrast, all of the H1HA-LEC and PBS-only injected mice lost up to 40% of their body weight and died by Day 18.

### Example 3

### Low Doses of Vaxfectin™-Formulated LECs can Protect Against Influenza Viral Challenge

The efficacy of influenza LEC vaccine was explored in two dose-response studies. In the first study, mice were vaccinated (Days 0 and 21; 10 mice per group) with either PBS-formulated H3HA-LEC (50 μg) or Vaxfectin™-formulated H3HA-LEC (50 μg) or Vaxfectin™-formulated MOD-H3HA-LEC (50, 10 and 2 μg). In addition, Vaxfectin™-formulated H3HA pDNA (100 μg) and PBS groups were included as positive and negative controls, respectively. At the end of the study (nine weeks after first vaccination) all mice in the homotypic (H3HA) groups survived viral challenge; challenge of mice in the PBS and H1HA-LEC groups resulted in 10% and 20% survival, respectively. No apparent weight loss was evident for animals in the homotypic vaccine groups except for the 2 μg Vaxfectin™-formulated MOD-H3HA-LEC where on average a maximum weight loss of about 7% was observed at Day 8.

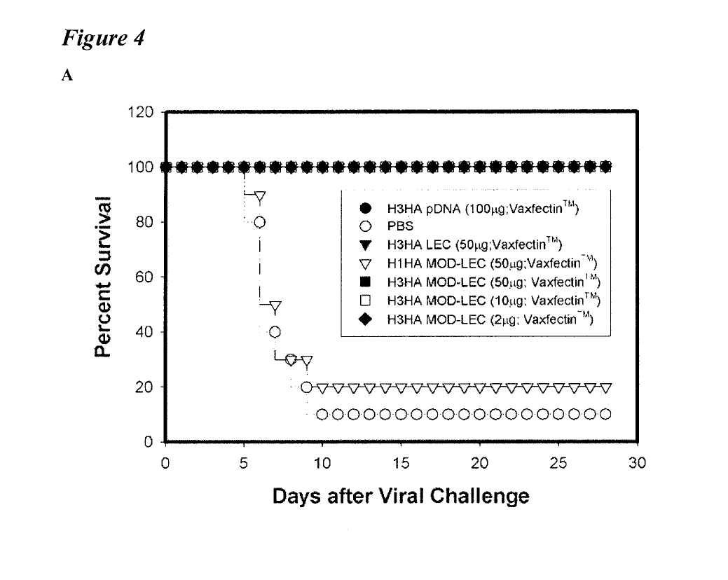

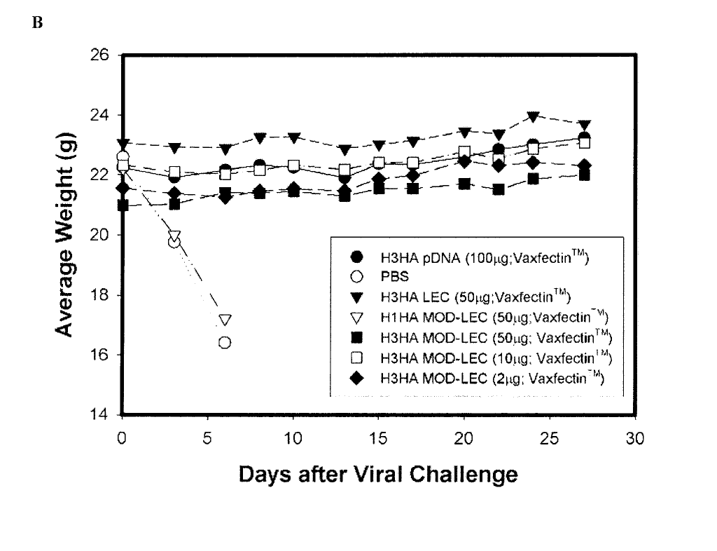

All animals in this last group recovered their weight by the end of the study. Animals in the heterotypic (H1HA) and negative control groups lost up to 25% of their body mass on average with animals surviving challenge recovering most of their body mass by the end of the study. Survival and body mass data for this study are summarized in FIG. 4. Survival at 28 days post-challenge showed statistical significance for H3HA-LEC and MOD-H3HA-LEC groups vs. either the PBS control or the H1HA-LEC.

### Example 4

### Single Administration of LEC Influenza Vaccine can be Protective Against Influenza Challenge

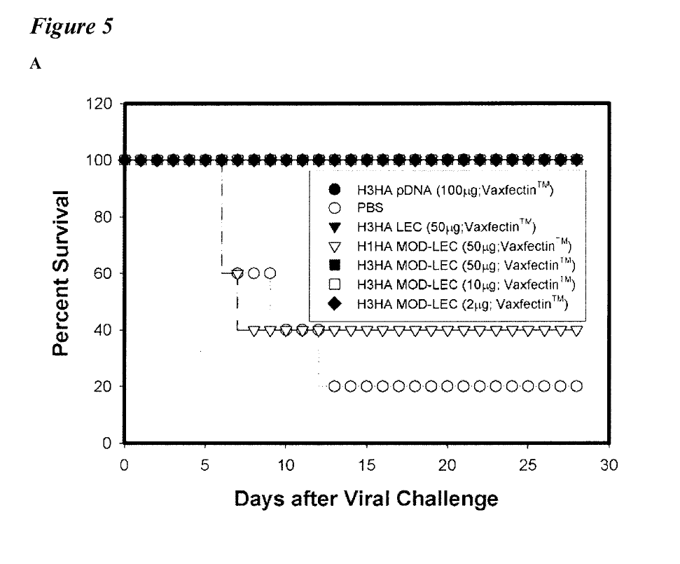

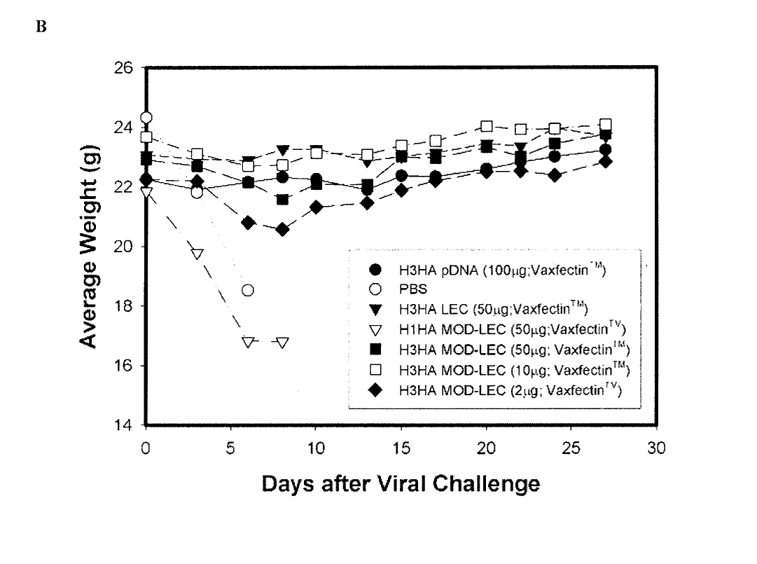

The second study used the same test and control groups described in 3.2 but mice received only one vaccination at Day 0. Viral challenge took place six weeks after vaccination; the study was terminated nine weeks after vaccination. Mice receiving as little as a single 2 μg injection of Vaxfectin™-formulated MOD-H3HA LEC were 100% protected from H3N2 virus challenge; 40% of mice in the MOD-H1HA-LEC group and 20% of animals in the PBS-only group survived the viral challenge. Survival and body mass data are presented in FIG. 5.

### Example 5

### Exemplary Results

The effectiveness of the PCR amplification conditions disclosed herein was supported by DNA sequence analysis of PCR-amplified LECs (data not shown). Additionally, the resultant LECs were tested in vitro for expression of the encoded influenza HA antigens. Western blot analysis showed the expression of proteins of the expected size and cross-reactivity (FIG. 2).

As described herein, lethal doses of a well characterized mouse-adapted influenza A/HK/8/68 (H3N2) virus were used to test the efficacy of LEC vaccination. The data in FIG. 3 indicate that homotypic LEC vaccination fully protects mice when administered at Days 0 and 21 at 50 μg per dose in PBS. Both survival and weight loss data for the homotypic LEC group (H3HA) are indistinguishable from the H3HA-pDNA positive control. On the other hand, none of the mice in the heterotypic LEC group and PBS groups survived the challenge showing lack of heterotypic cross-protection and confirming the lethality of the viral dose used, respectively.

The effect of using exonuclease resistant PCR-primers to produce an LEC of the disclosure was tested by using oligonucleotide PCR primers where the two 5′-most residues were phosphorothioates. The resulting LEC (MOD-LECs) was formulated with Vaxfectin™ to benefit from the adjuvant effects of this cationic lipid system. Mice were vaccinated with Vaxfectin™-formulated MOD-LECs using doses ranging from 50 μg to 2 μg. Moreover, the efficacy of vaccination using a single dose of the Vaxfectin™-formulated MOD-LEC was tested.

Survival and weight loss data shown in FIG. 4 indicate that two administrations of the LEC vaccine completely protect mice from the viral challenge. Remarkably, single administration of the LEC H3HA vaccine at the lowest dose tested (2 μg) was sufficient to confer protection to viral challenge. Mice appeared active and healthy; the only indication of disease in this group was a slight (<10%), non-statistically significant, decrease in average body mass by Day 9. All the animals in this group recovered their weight by the end of the study.

The data therefore demonstrate the feasibility of using linear DNA-based vaccines to protect against a lethal dose of influenza A virus. Efficacy of Vaxfectin™-formulated LECs at low doses is consistent with previously published data showing enhanced immune responses of cationic lipid-formulated DNA. Efficacy and ease of manufacture support further evaluation of LEC-based influenza vaccines as a promising approach to rapid vaccine development.

All references cited herein, including patents, patent applications, and publications, are hereby incorporated by reference in their entireties, whether previously specifically incorporated or not.

Having now fully provided the instant disclosure, it will be appreciated by those skilled in the art that the same can be performed within a wide range of equivalent parameters, concentrations, and conditions without departing from the spirit and scope of the disclosure and without undue experimentation.

While the disclosure has been described in connection with specific embodiments thereof, it will be understood that it is capable of further modifications. This application is intended to cover any variations, uses, or adaptations of the disclosure following, in general, the disclosed principles and including such departures from the disclosure as come within known or customary practice within the art to which the disclosure pertains and as may be applied to the essential features hereinbefore set forth.

1. A linear expression cassette (LEC) comprising i) an expression codon optimized DNA coding sequence operably linked to a promoter and ii) terminal primer sequences for polymerase chain reaction (PCR) mediated amplification of the LEC.

2. A linear expression cassette (LEC) comprising i) an expression codon optimized DNA coding sequence, encoding an antigen, or fragment thereof, which produces an immune response upon expression in an animal or human subject, operably linked to a promoter and ii) terminal primer sequences for polymerase chain reaction (PCR) mediated amplification of the LEC.

3. The LEC of claim 2 wherein the antigen, or fragment thereof, is that of a pathogen and produces a protective immune response upon expression in the animal or human subject.

4. The LEC of claim 1 wherein the coding sequence encodes a patient specific anti-idiotype product, a fusion protein comprising an antigen or fragment thereof, or a concatemer of antigens.

5. A linear expression cassette (LEC) comprising i) an expression codon optimized DNA coding sequence, encoding a diagnostic reagent, operably linked to a promoter, and ii) terminal primer sequences for polymerase chain reaction (PCR) mediated amplification of the LEC.

6. The LEC of claim 2 wherein the antigen, or fragment thereof, comprises a secretion signal.

7. The LEC of claim 1 wherein the promoter is supercoiling independent, stronger than a CMV promoter on a linear template in a eukaryotic cell, weaker than a CMV promoter on a linear template in a eukaryotic cell, or comprises one or more regulatory elements.

8. The LEC of claim 1 further comprising a non-coding sequence between the promoter and the coding sequence.

9. The LEC of claim 1 further comprising a 5′ moiety that resists exonuclease degradation.

10. The LEC of claim 1 further comprising a proteinaceous nuclear localization signal (NLS) or
a nucleic acid moiety that directs delivery of the LEC to the nucleus or a mitochondrion of a cell.

11. The LEC of claim 1 further comprising one or more phosphorothioate linkages in the DNA backbone.

12. A composition comprising a particle, bead, polymer or suspendable solid support removably associated with an LEC comprising i) a codon optimized DNA coding sequence operably linked to a promoter and ii) terminal primer sequences for polymerase chain reaction (PCR) mediated amplification of the LEC.

13. A composition comprising a lipid vesicle carrier and an expression optimized LEC comprising i) a codon optimized DNA coding sequence operably linked to a promoter and ii) terminal primer sequences for polymerase chain reaction (PCR) mediated amplification of the LEC.

14. The composition of claim 13, wherein said lipid vesicle carrier comprises a cationic lipid and a neutral lipid.

15. The composition of claim 14 wherein said cationic lipid is VC1052 and said neutral lipid is DPyPE in a molar ratio of about 1:1.

16. The composition of claim 14 wherein the molar ratio of LEC to total lipid is from about 8:1 to about 1:8 or higher proportion of lipid.

17. The composition of claim 16 wherein the vesicle is a liposome associated with said LEC.

18. A unit dose formulation or composition comprising
about 1 nanogram to about 5 milligram of a linear expression cassette (LEC) comprising i) a codon optimized DNA coding sequence operably linked to a promoter and ii) terminal primer sequences for polymerase chain reaction (PCR) mediated amplification of the LEC; and
a pharmaceutically acceptable carrier or excipient, and optionally an adjuvant.

19. A linear expression cassette (LEC) produced by PCR amplification of a nucleic acid template comprising i) an expression codon optimized DNA coding sequence operably linked to a promoter and ii) terminal primer sequences for polymerase chain reaction (PCR).

20-23. (canceled)

24. An immunostimulatory composition comprising:
i) a linear expression cassette (LEC) produced by polymerase chain reaction (PCR) amplification of a nucleic acid template comprising a) an expression codon optimized DNA coding sequence operably linked to a promoter and b) terminal primer sequences for PCR; and
ii) a linear cassette (LC) comprising i) an immunostimulatory motif which produces an immune response upon being introduced in an animal or human subject, operably linked to a promoter and ii) terminal primer sequences for polymerase chain reaction (PCR) mediated amplification of the LC.

25. A linear expression cassette (LEC) comprising i) a codon optimized DNA coding sequence, encoding an antigen, or fragment thereof, which produces an immune response upon expression in an animal or human subject, operably linked to a promoter and ii) terminal primer sequences for polymerase chain reaction (PCR) mediated amplification of the LEC, wherein said LEC further comprises one or more phosphorothioate linkages in the DNA backbone.


SEQUENCE LISTING (2 sequences)

SEQ ID NO: 1 - DNA, 30 bp, Artificial Sequence
  Forward oligonucleotide primer for PCR amplification of LEC DNA
tggccattgcatacgttgtatccatatcat

SEQ ID NO: 2 - DNA, 30 bp, Artificial Sequence
  Reverse oligonucleotide primer for PCR amplification of LEC DNA
agtcagtgagcgaggaagcggaagagtacc